(19) United States
(12) Patent Application Publication
(10) Pub. No.: US 2002/0147353 A1
(43) Pub. Date: Oct. 10, 2002

(54) Novel flavonoids

(75) Inventors: Willem J.F. Van Der Vijgh, Diemen (NL); Aalt Bask, Nijkerk (NL); Frederique A.A. Van Acker, Amsterdam (NL); Wiro M.P.B. Menge, Arnhem (NL); Guido R.M.M. Haenen, Eijsden (NL)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Vereniging christelijk wetenschappelijk anderwijs, Amsterdam (NL)

(21) Appl. No.: 10/102,733

(22) Filed: Mar. 22, 2002

Related U.S. Application Data
(63) Continuation of application No. PCT/NL00/00649, filed on Sep. 23, 2000.

(30) Foreign Application Priority Data
Sep. 23, 1999 (NL) 1013123

(51) Int. Cl. C07H 17/00; C07D 311/26; C07D 311/74

(52) U.S. Cl. 549/403; 536/8

(57) ABSTRACT

Novel flavonoids of formula (I)

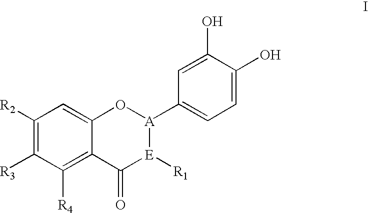

where A and E form together a C—C or C═C bond; R₁, R₂, R₃, and R₄ are H, OH, O(CH2)ₙ— aromatic group, n=0-8; O(CH₂)ₙ N(CH₃)_{q} with n=0-8, q=0-3; O(CH₂)ₙ OH with n=1-8; O(CH₂)ₙ-halide with n=1-8; O(CH₂)ₙ COOH with n=0-8; O(CH₂)ₙ COOR′ with n=0-8 and R′ is C₁-C₈ alkyl or an aromatic group; O(CH₂)ₙ CONH R″ with n=0-8 and R″ is C₁-C₈ alkyl or an aromatic group, and sugars in mono-, di- or trimeric form or analogues thereof, with the proviso that R₁ is not H, at least two of R₂, R₃ and R₄ are H, and at most one of R₁, R₂, R₃ and R₄ is OH, are useful for the treatment of drug-induced toxicity, doxorubicin-induced cardiotoxicity, free radical mediated diseases, lung diseases, cancer, diabetes mellitus, cardiovascular disease, or arteriosclerosis.

This is a continuation application of PCT/NL00/00649 filed on Sep. 13, 2000, which PCT application claims priority of Dutch patent application number NL 1013123 filed on Sep. 23, 1999, both herein incorporated by reference.

### FIELD OF THE INVENTION

The invention relates to novel flavonoids, a method for obtaining flavonoids, and to the use of the flavonoids as a medicament.

In this application, the term flavonoids and the nomenclature thereof is used as is defined in S.A.B.E. van den Acker, Chem. Res. Toxicol. 1996, 9, 1305-1312, herein incorporated under reference.

### SUMMARY OF THE INVENTION

The new compounds according to the invention are defined by formula I,

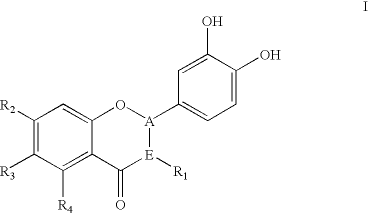

wherein:

A and E form together a C—C or C═C bond,

R1, R2, R3, and R4 are chosen from:

H,

OH, and from the substituents,

O(CH₂)ₙ-aromatic group, n=0-8,

O(CH₂)ₙ N(CH₃)_{q} with n=0-8, q=0-3,

O(CH₂)ₙ OH with n=1-8,

O(CH₂)ₙ-halide with n=1-8,

O(CH₂)ₙ COOH with n=0-8,

O(CH₂)ₙ COOR′ with n=0-8 and R′ is C₁-C₈ alkyl or an aromatic group,

O(CH₂)ₙ CONH R″ with n=0-8 and R″ is C₁-C₈ alkyl or an aromatic group,

sugars in mono-, di- or trimeric form or analogues thereof, with the proviso that:

R1 is not H,

at least two of R2, R3 and R4 are H, and

at most one of R1, R2, R3 and R4 is OH.

According to the above, a substituent is defined as any moiety except H or OH, covalently linked to a carbon atom of the backbone of the flavanoid compound.

It was found that the novel flavonoids according to the invention have an excellent antioxidant activity and are potent candidates for use in a medicament, wherein the antioxidant activity is required. In the novel flavonoids the A ring, i.e. the ring of moieties R2, R3 and R4, has at most one substituent on the 5-position (R4), 6-position (R3) or 7-position (R2); further, said flavonoids have a substituent R1, as defined above (not being H), on the 3-position. In the art, it is believed that the antioxidant activity increases with the number of available OH groups in the A and B ring of the flavonoid (Cao G. et al., Free Rad Biol. Med. 22, 749-760 (1997) and Chen Z. Y., Chem. Phys. Lipids. 79, 157-163 (1996)). In contrast, it was found that the compounds according to the invention had an improved antioxidant activity compared to the known compound fisetine, wherein both R1 and R2 are OH (S.A.B.E. van Acker et al., Free Rad. Biol. Med. 20, 331-342, 1996). It is now postulated that the presence of a hydroxy group in the A ring, combined with a hydroxy group in the C ring, leads to a molecule having pro-oxidant activity instead of an antioxidant activity, i.e. such a compound would rather induce the formation of radicals than inhibiting said formation. Examples of such compounds having an OH group on the 3′-position and an OH group as single substituent in the A ring is e.g. fisetine (S.A.B.E. van Acker et al, supra).

In defining the structural features that determine the antioxidant properties of flavonoids (S.A.B.E. van Acker, et al., supra, and S.A.B.E. van Acker, et al., Chem. Res. Tox. 9, 1305-1312 (1996)), it was found that a catechol moiety on ring B is required for scavenging activity, and that substitution in the A ring is allowed but influences the antioxidant activity. Moreover it was found that a OH-group in position 3 in combination with a C₉-C₃ double bond positively affected the antioxidant action of flavonoid. Therefore, A and E preferably form a C═C bond, although a C—C bond is also possible. These considerations were the basis for the synthesis of the new flavonoids according to the invention with variations in the A ring (substituents R2, R3 and R4) and in position C3 (substituent R1). In this way novel and more active compounds were obtained. In our previous work 7-monohydroxyethylrutoside (monoHER) appeared to be an interesting antioxidant (e.g. S.A.B.E. van Acker, et al., Br. J. Pharmacol. 115, 1260-1264 (1995)). It appeared however that the antioxidant activity of the compound was still too low and therefore the dose is expected to be too high to be practical in vivo. The optimisation of flavonoids according to the invention has led to novel effective antioxidants. The new compounds were indeed extremely effective inhibitors of the iron/ascorbate induced lipid peroxidation, as will be explained in more detail below. In more complex models, i.e. the in vitro and in vivo protection of the free radical mediated cardiotoxicity of doxorubicin, the compounds were shown to be active as well, see below, indicating that the compounds are very potent candidates as drug against free radical mediated diseases.

Because of the possible use of these compounds as adjuvants in chemotherapy, i.e. to protect against the side effects of cytostatics, such as doxorubicin, it was checked whether the compounds had an influence on the antitumor activity of doxorubicin.

It was further found that the newly synthesized compounds inhibited the growth of a tumor cell line (OVCAR-3) and may be candidates for a potent antitumor drug.

Advantageously the compound according to the invention comprises at least one substituent, as listed above.

Regarding the substituents in the A-ring, the compound according to the present invention preferably comprises a substituent on the 7-position (R2), implicating that R3 and R4 are both H. Substitution of the 7-position leads to flavonoids of excellent antioxidant activity. Further, R1 in the C-ring is preferably a substituent.

The term “Sugars” is well known in the art. Usually, sugars are defined as carbohydrates with formula (CH₂O)ₙ, n being 5-7, like glucose, fructose, mannose, or sugar analogues like aminosugars, artificial starch, carboxysugars or deoxysugars.

Preferably, the sugar is a C₅-C₇ sugar, preferably a mono-, di- or trisaccharide, i.e. a frequently occurring natural sugar.

The sugar is preferably coupled to the E moiety (the C₃ position) of the flavonoid, as O-linked sugars; in particular saccharides have shown to have an improved bioavailability in vivo.

Further, the sugars may advantageously be acetylated especially when metabolically more stable compounds are desired. Acetylated sugars, particularly saccharides, are metabolically more stable than their non-acetylated counterparts, as the acetylation prevents undesired sulfatation as well as the conjugation reaction mediated by e.g. glucuronidase.

Preferably, the compound according to the invention is chosen from the following group, wherein reference is made to formula I and wherein A and E form a C═C bond, and R3 and R4 are both H. R2 and R1 are preferably as follows:

R2═H and RL═OH,

R2═H and R1═OCH₃,

R2═H and R1═OCH₂CH₂OH,

R2═H and R1═OCH₂COOH,

R2═H and R1═O(CH₂)₃N(CH₃)₃,

R2═H and R1═O(CH₂)₃N⁺(CH₃)₃,

R2═H and R1═O(CH₂)₆N⁺(CH₃)₃,

R2═H and R1═O(CH₂)₈N⁺(CH₃)₃,

R2═H and R1=optionally acetylated Orutinose,

R2═H and R1=optionally acetylated Oglucose,

R2═OH and R1═OCH₃,

R2═OH and R1═OCH₂CH₂OH,

R2═OH and R1═O(CH₂)₃N⁻(CH₃)₃, or

R2═OCH₂CH₂OH and R1═OH.

Most preferably, R2 is H and R1 is an O-glucose. It could be shown that this compound has an improved bio-availability and therewith it is expected to be an active compound regarding antioxidant activities, already effective at relatively low doses.

Preferably, the compound according to the invention is synthetic or semi-synthetic, i.e. that at least part of the synthesis has been done by chemical engineering. Preferably, the compound is fully synthetic. The advantage of a synthetic compound is the fact that any impurities that may be present in a crude extract from a natural source are absent.

The invention also relates to a method for the preparation of a compound of formula Ia,

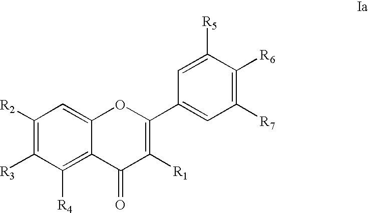

wherein

R1, R2, R3 and R4, R5, R6 and R7 are chosen from: H,

OH, and from the substituents

O(CH₂)ₙ-aromatic group, n=0-8,

O(CH₂)ₙ N(CH₃)_{q} with n=0-8, q=0-3,

O(CH₂)ₙ OH with n=1-8,

O(CH₂)ₙ-halide with n=1-8,

O(CH₂)ₙ COOH with n=0-8,

O(CH₂)ₙ COOR′ with n=0-8 and R′ is C₁-Cg alkyl or an aromatic group,

O(CH₂)ₙ CONH R″ with n=0-8 and R″ is C₁-C₈ alkyl or an aromatic group, and

sugars in mono-, di- or trimeric form or analogues thereof, wherein at least one of R1-R7 is chosen from the above substituents, and at least one other of R1-R7 is OH, by reacting a compound of formula II:

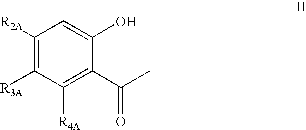

with a compound of formula III:

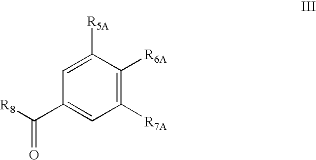

to form a compound with formula IV:

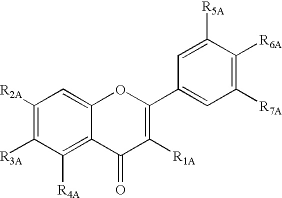

wherein R2A-R7A are, independently from each other, H, OH or the substituent R2-R7, respectively, as defined above,

R₈ is C₁-C₈ alkoxy, preferably OCH₂CH₃, or H,

R1A is H or OH, and

at least two of R1A of formula IV, and of R2A-R7A of formulas II and III being OH,

comprising the steps of:

a) protecting of at least one of the OH groups of R1A-R7A of the compounds of form II, form III and form IV with a protecting group, leaving at least one of the OH groups of R1A-R7A unprotected,

b) substituting of at least one of the unprotected OH groups by any of the substituents as defined above,

c) deprotecting at least one of the OH groups, protected in step a).

As starting materials, hydroxyacetophenone (formula II) and a benzaldehyde (formula III, wherein R8═H) or a lower benzoate ester (formula III, wherein R8═C₁-C₈ alkoxy) are used. Further, one or more R2A-R7A should contain an OH-group, when in the end product (formula I or formula Ia) the corresponding R2-R7 comprises a substituent. It is however also possible to have one or more of the substituents already present at the corresponding positions on the starting materials instead of the corresponding OH-group.

It is to be noted that the position of the groups R1-R7 in the end product corresponds to the position of R1A-R7A, respectively, in the starting and intermediate compounds.

In a subsequent substitution step, a free OH-group on one or more of the positions R1A-R7A is substituted by the desired substituent, the possible substitution reactions of which are known in the art, some advantageous reactions being discussed below. The OH-groups on the R2A-R7A positions that are not to be substituted are protected before the reaction of the starting materials. “Protection” is understood to be a chemical reaction, wherein the hydroxy-group is temporarily and reversibly changed or chaperoned, leading to a protected OH-group that is resistant against the later substitution reaction.

The protective groups present in de 2-hydroxyacetophenones and on the benzaldehydes and benzoate esters may be benzyl or substituted benzyl, methyl, alkyl or cycloalkyl, acetoyl or benzoyl or substituted silyl groups, and are e.g. introduced via reaction of the appropriate halide in the presence of a base in a polar solvent, typically benzyl bromide and potassium carbonate in acetone.

Hydroxy acetophenones, of which the relevant OH-groups are protected as indicated above, e.g. in the form of phenoxy groups, can be reacted with similarly protected benzaldehydes by an aldol condensation (Pfister J. R. et al., J. Med. Chem, 23, 335-338, 1980) leading to intermediate chalcones. By e.g. oxidative cyclisation of the chalcones with hydrogen peroxide via the Algar-Flyn-Oyamada reaction (Algar, J., Flyn, J. P., Proc. Roy. Irish Acad., sect. B.42, 1-8 (1934), Oyamada, T. J., Chem., Soc. Jpn., 55, 1256-1261 (1934), flavonols according to formula IV (i.e. having an OH-group at the R1 position), are obtained. Solvents which can be used for this reaction are lower alcohols, such as methanol and ethanol, dioxane or other cyclic ethers, water or a mixed solvent of one or more of the above mentioned alcohols, dioxane and water.

In addition to the hydroxy groups on positions R2A-R4A on 2-hydroxyacetophenone and on positions R5A-R7A on the benzaldehyde, the OH-group at the R1 position of the obtained flavonol can function as a substrate for a substitution or protection reaction.

E.g. in case R1 in the end product is OH, said OH is to be protected in the intermediate flavonol of formula IV, obtained by the reaction of the hydroxyacetophenone and the benzaldehyde, when a substitution reaction on the R2A, R3A or R4A position is followed.

The reaction of 2-hydroxyacetophenone, of which the relevant OH-groups are protected, with accordingly protected lower benzoate esters can be done in the presence of a strong base such as alkali alkoxides or alkali amides in solvents such as a lower alcohol or dialkylethers or cyclic ethers, typically lithium diisopropylamide in tetrahydrofurane, resulting in the intermediate 1,3-propanediones (Baker, W. J., Chem. Soc. 1381-1389 (1933), and Robinson, R., Venkataraman, K. A., J. Chem. Soc., 2344-2348 (1926). Said 1,3-propanediones are preferably not isolated but cyclised directly to the intermediate flavones according to formula IV, wherein R1 is H. This may be achieved via an acid catalyzed cyclisation in lower alcohols using strong mineral acid or strong acid cation exchange resins, typically isopropanol and Dowex W50-X8.

Substituents for the 3,5,6 and 7-position, substituting for OH on R1A, R4A, R3A and R2A respectively of formula IV can be introduced by reaction with an alkyl halide (RX) in the presence of a base, typically alkali metal hydroxides or alkali metal carbonates in a polar solvent, such as dimethyl formamide (DMF) or ethanol or acetone. Alternatively, the intermediate flavonol or flavone is treated with a cyclic alkyl carbonate and alkali carbonate in the absence of solvent. As another possibility, the intermediate flavonol or flavone is obtained via reaction with an alkyl dihalide, followed by reaction with ammonia, mono-, di- or tri-substituted amines. Alternatively, the intermediate flavone or flavonol is treated with a peracetylated-alfa-bromosaccharide and a silver salt (Horhammer et al., Chem. Ber. 99, 1384-1387 (1966)), typically silver oxide, in pyridine. The flavone and flavonol derivatives of formula I or Ia are obtained by simultaneous or stepwise removal of the protecting groups. For example, if the protective groups are benzyl groups, they are removed by reaction with hydrogen using a metal catalyst, or can be removed using boron trichloride or hydrochloric acid in acetic acid. The acetyl or benzoyl protective groups are removed by reaction with alkali metal hydroxide or alkali metal alkoxide in a lower alcohol or water or a mixture of said alcohol and water. The methyl or alkyl groups are removed by Lewis acids, such as aluminum chloride or boron trichloride.

When the end product should contain two or more different substituents, one or more of the said substituents can already be present on the 2-hydroxyacetophenone, the benzaldehyde or the lower benzoate ester, as indicated above. However, in an attractive embodiment of the present invention, the positions on the starting materials corresponding to those of the end product containing those substituents, are protected by different protecting groups that can selectively be removed as is claimed in appended claim 11. In a first substitution reaction one or more free OH-groups are then substituted by the desired substituent, followed by deprotection of one or more, but not all, protected OH-groups by a selective reaction resulting in deprotection of only the desired OH-groups, followed by substitution of those newly deprotected OH-groups by another substituent; this is followed by a next (selective) deprotection step, resulting in OH-groups, available for a next substitution step. In this way, a flavonoid compound according to the invention can be produced, comprising 2-6 different substituents on the R1A-R7A positions. In case the R1A position is to be substituted in this way, the OH-group on position R1A of the flavonol of formula IV can be protected/substituted once the flavonol is obtained from an optionally protected 2-hydroxyacetophenone and an optionally protected benzaldehyde.

In another advantageous embodiment, the method according to the invention comprises, after the last deprotection step, a final substitution step, substituting the OH-group or OH-groups, deprotected at the last deprotection step, by another substituent. When the final step in the method as described above is a deprotection step, the resulting compound will comprise at least one OH-group on the R1-R7 positions. By a final substitution step, this OH-group or groups can be substituted as well, resulting in a final flavonoid of which all previously present OH-groups are substituted. In this way, all R1A-R7A positions may comprise a substituent.

Preferably, the method according to the present invention is used for the preparation of a novel flavonoid compound according to the present invention, wherein A-E form together a C═C bond. For this, the compound of formula III is a benzaldehyde (R8═H) and one or two substitution steps are needed.

The above mentioned method is therefore also very suitable for the preparation of the novel compounds of the present invention. For this, the 2-hydroxyacetophenone is to be chosen such, that at most one of R2A, R3A and R4A, preferably R2A, is OH, and at least two thereof are H, and wherein the substituents are chosen such, that in the end product at least two of R2, R3 and R4 are H, and at most one of R1, R2, R3 and R4 is OH.

It is to be noted that the above mentioned reactions are given as illustration and are not intended to be limitative. The skilled person will immediately recognize other suitable reactions to obtain the desired flavonoids as end products, starting from hydroxyacetophenones and benzaldehyde or benzoate ester.

In another aspect the invention relates to the use of a novel compound of the present invention according to formula I in a medicament.

A survey of registered drugs reveals that many drugs have moderate antioxidant properties that might contribute to their therapeutic effectiveness (A. Bast, Drugs News Perspect. 7, 465-472 (1994)). The antioxidant activity is merely a side-effect of these drugs. Because free radicals play a role in many pathologies, there is a need for more active antioxidant drugs.

Long term effects of free radicals can be discerned in the complications involved in chronic diseases like arteriosclerosis, diabetes, chronic obstructive pulmonary disease (COPD) and drug-induced toxicities like the anthracycline-induced cardiotoxicity and the bleomycine-induced lung toxicity.

Compounds according to formulas 1 and la were synthesized as potent antioxidants that can be employed as therapeutic agents. A rational strategy in the selection of new structures that were synthesized, was possible as a discussion regarding the structure-activity relationship for the antioxidant action of flavonoids has been reported (S.A.B.E. van Acker et al., Free Rad. Biol. Med. 20, 331-342 (1996); S.A.B.E. van Acker et al., Chem. Res. Tox. 9, 1305-1312 (1996)).

Further the new flavonoid compounds with improved antioxidant properties as well as known flavonoids were tested in several pharmacological and toxicological assays. Their antioxidant activities, their cardioprotective properties and antitumor activities were determined in vitro and it was checked whether the compounds had any possible toxic properties in hepatocytes. Based on these data, compounds were selected for further investigations in vivo. After in vivo toxicology studies, a further selection was made to study its cardioprotective properties in vivo using telemetry. One of the selected compounds according to the invention, wherein R2, R3 and R4 are H and R1 is Oglucose, provided complete protection against doxorubicin-induced cardiotoxicity with a dose five times lower than the dose needed for monoHER.

Doxorubicin is a very effective antitumor agent used in the treatment of various solid tumors. Its clinical use is largely limited by the occurrence of a cumulative dose-related cardiotoxicity, which manifests itself as congestive heart failure. This observed cardiotoxicity is believed to be mainly caused by free (oxygen) radicals. Therefore flavonoids according to formula I or Ia, preferably according to formula I are very well suitable as active compounds in a medicament against doxorubicin-induced cardiotoxicity, which will be explained in more detail below.

Based on the results of these assays, which will be discussed below, the said compounds are good candidates as active compound in a medicament for treatment of a condition, wherein antioxidant activity of the medicament is needed. Preferably, said flavonoids are used in a medicament or food supplement for treatment and prevention of a condition, chosen from the group, consisting of: drug induced toxicity, including anthracycline-induced cardiomyopathy and vascular damage, doxorubicin induced cardiotoxicity, free radical mediated diseases, lung disease and cancer, in particular as cytostatic agent. In this respect, the term “food supplement” is to be regarded as a medicament. Preferably, one or more compounds according to formula I are used as active compound in such a medicament. Furthermore, the compound of formula I can be used as active compound in a medicament for the treatment of diabetes mellitus, in particular vascular and neuronal complications thereof, and cardiovascular diseases, especially arteriosclerosis.

### BRIEF DESCRIPTION OF THE DRAWINGS

The invention is illustrated with the accompanying drawing, wherein:

FIG. 1 depicts a schematic synthesis scheme for the preparation of flavonoids according to the present invention, wherein in FIG. 1a the formation of an intermediate flavone or flavonol from a 2-hydroxyacetophenone with a benzaldehyde or benzoate ester is depicted, and in FIG. 1b substitution at the 3 position and the protection is shown;

FIG. 2 shows the prevention of in vivo doxorubicin-induced cardiotoxicity by the i.p. administration of a compound according to the present invention, wherein R1═O glucose and R2, R3 and R4 are H.

### EXAMPLES

Materials and Methods

Elemental analysis were performed for C, H, N (Department of Microanalysis, Groningen University, The Netherlands. ¹H NMR and ¹³C NMR spectra were recorded on a Bruker AC-200 (200 MHz) spectrometer. Chemical shifts for ¹H and ¹³C NMR are given in ppm (o) relative to tetramethylsilane (TMS) as internal standard. Melting points (not corrected) were measured on an Electrothermal AI-9100 apparatus. THF was dried over LiAlH₄ and distilled before use;

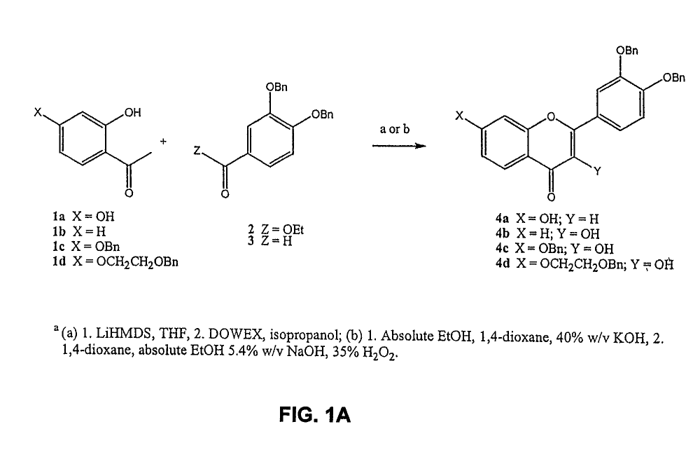

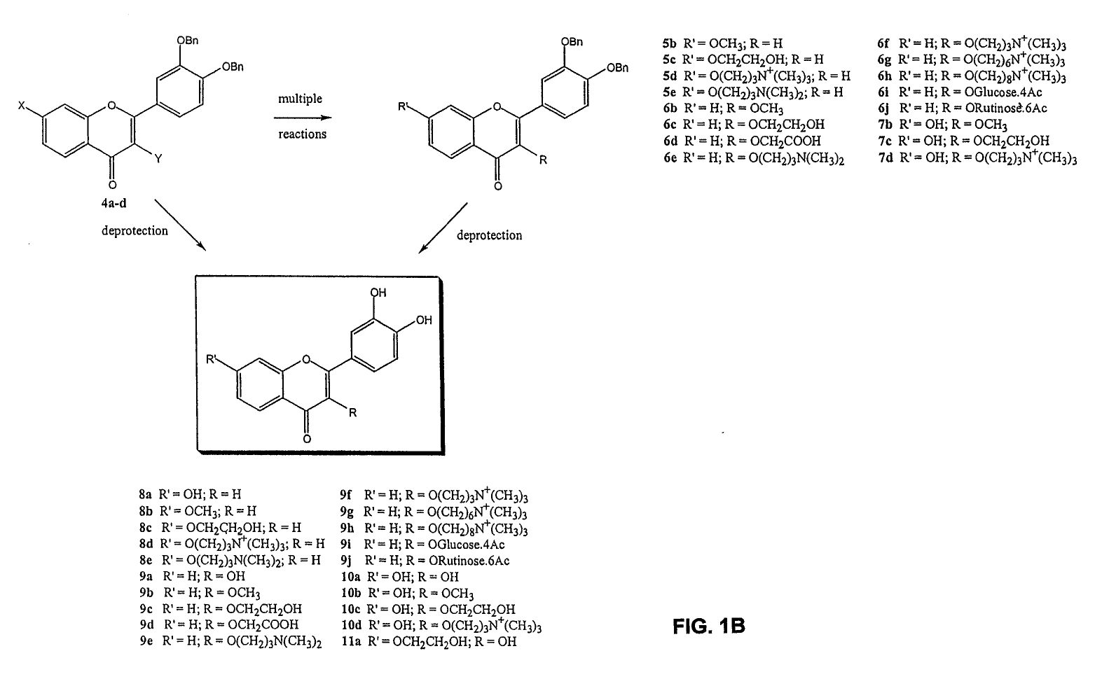

The numbers between brackets in bold refer to the compound as is indicated in FIG. 1.

Syntheses

3′,4′-dibenzyloxy-7-hydroxyflavone (4a)

A stirred suspension of 60 mmol of ethyl 3,4-dihydroxybenzoate, 41 g of K₂CO₃ and 15 mL (0.126 mol) of benzylbromide in 250 mL of acetone was heated to reflux for 15 h. The solvent was evaporated and the residue was suspended in 150 mL of H₂O and extracted with CH₂Cl₂ (2×150 mL). The combined organic layers were dried over sodium sulfate and concentrated under reduced pressure. The crude product was purified by column chromatography (CH₂Cl₂/hexane, 1:1 (v/v)) to give 19.4 g (89%) of ethyl-3,4-dibenzyloxybenzoate (2); mp 68-69° C.

32.28 g (200 mmol) 1,1,1,3,3,3-hexamethyldisilazan (HMDS) was dissolved in 250 mL dry THF and cooled to −60° C. under an atmosphere of dry nitrogen. 125 mL (200 mmol) butyllithium (1.6 M in hexane) was added dropwise followed by 50 mmol of 2,4-dihydroxyacetophenone (1a) in 100 mL dry THF and stirred for 45 min at −30° C. The reaction mixture was cooled to −60° C. and 50 mmol of 2 in 100 mL dry THF was added dropwise. This mixture was stirred for another 45 min at −60° C. and at room temperature overnight. The reaction mixture was poured on ice and acidified with 3.0 M HCl. The THF was removed under reduced pressure and extracted with chloroform (2×150 mL). The chloroform layers were dried over sodium sulfate and concentrated in vacuo. The crude 1-(2,4-dihydroxyphenyl)-3-(3,4-dibenzyloxyphenyl)-1,3-propanedione was dissolved in 100 mL of isopropanol, 25 g DOWEX W50x8 (H⁻ form) was added and heated under reflux for 16 h under an atmosphere of dry nitrogen. The solids were filtered and the product was dissolved in DMF and filtered to remove the Dowex. The DMF was removed under reduced pressure to give 11.0 g (49%) 4a as a white solid; mp 245.4-245.8° C. ¹H NMR (DMSO): δ 5.25 (s, 2H, OCH₂Ph), 5.29 (s, 2H, OCH₂Ph), 6.87 (s, 1H, C3H), 6.91 (dd, 2H, J=9 Hz, 2 Hz, C5′H), 7.0 (d, 1H, J=2 Hz, C₈H), 7.21 (d, J=8 Hz, C6H), 7.3-7.6 (m, 10H, OCH₂Ph×2), 7.64 (dd, 1H, J=7 Hz, 2 Hz, C6′H), 7.70 (t, J=2 Hz, C2′H), 7.86 (d, 1H, J=9 Hz, CSH), 10.9 (br s, 1H, OH).

3′,4′-dibenzyloxy-3-hydroxyflavone (4b)

A suspension of 31.4 mmol 3,4-dibenzyloxybenzaldehyde (3) and 31.4 mmol 2-hydroxyacetophenone (lb) in 80 mL ethanol and 50 mL dioxane was cooled to 10° C. and 25 mL 40% w/v KOH solution was added dropwise. The reaction mixture was stirred for 66 h at room temperature. 400 mL CH₂Cl₂ was added and the organic layer was washed with H₂O (3×50 mL), dried over sodium sulfate and concentrated in vacuo.

The oily residue was dissolved in 110 mL dioxane and 300 mL ethanol, and 100 mL 5.4% (w/v) NaOH solution. 11.4 mL 35% H₂O₂ was added dropwise. The reaction mixture was stirred on ice for 2 h and subsequently at room temperature overnight, resulting in a yellow suspension. After acidification with 100 mL 2 M HCl the precipitate was filtered and washed with 500 mL H₂O. The product was recrystallized from ethanol to give 7.0 g (49%) of a light yellow solid; mp @. ¹H NMR (CDCl₃): δ 5.22 (s, 2H, OCH₂Ph), 5.25 (s, 2H, OCH₂Ph), 7.01 (d, J=8 Hz, C5′H), 7.08 (br s, 1H, C3OH), 7.3-7.5 (m, 12H, OCH₂Ph×2+C6H+C8H), 7.64 (dt, 1H, J=7 Hz, 1.5 Hz, C7H), 7.82 (dd, 1H, J=9 Hz, 2 Hz, C6′H), 7.92 (d, 1H, J=2 Hz, C2′H), 8.20 (dd, 1H, J=8 Hz, 2 Hz, C5H).

3-hydroxy-7,3′,4′-tribenzyloxyflavone (4c)

3,4-dibenzyloxybenzaldehyde (3) was reacted with 4-benzyloxy-2-hydroxyacetophenone (lc) in a similar way as described for 4b. The brown oil resulting from step 1 was crystallized from ethanol/chloroform (5:1) to give 1.8 g (42%); mp 120.3-121.3° C. In the second step the product was extracted with CH₂Cl₂ (2×100 mL) The organic layers were dried over sodiumsulfate and evaporated under reduced pressure. The product was crystallized form ethanol/CH₂Cl₂ (100 mL; 2:1) to give 1.82 g (41%) of 4c. Mp 160.6-161.2° C. ¹H NMR (CDCl₃): δ 5.16 (s, 2H, C7OCH₂Ph), 5.24 (s, 4H, C3′OCH₂Ph+C4′OCH₂Ph), 6.9 (br s, 1H, C3OH), 6.96-7.04 (m, 3H, C5′H+C6H+C8H), 7.23-7.47 (m, 15H, 3× OCH₂Ph), 7.77 (dd, 1H, J=9 Hz, 2 Hz, C6′H), 7.87 (d, 1H, J=2 Hz, C2′H), 8.10 (d, 1H, J=9 Hz, C5H).

7-(2-benzyloxyethoxy)-3-hydroxy-3′,4′-dibenzyloxyflavone (4d)

A mixture of 100 mmol benzylbromide and 2.0 mmol tetraethylammoniumiodine was heated to 150° C. for 15 min under an atmosphere of dry nitrogen. 160 mmol ethylenecarbonate was added in 4 equal portions with 30 min intervals and this was heated to 150° C. for 16 h under an atmosphere of dry nitrogen. The desired β-bromoethylbenzylether was isolated after distillation under reduced pressure. Yield: 16.27 g (76%) of a colourless liquid; Bp 75° C. ¹H NMR (CDCl₃): δ 3.45 (t, 2H, J=6 Hz, OCH₂CH₂Br), 3.76 (t, 2H, J=5.83 Hz, OCH₂CH₂Br), 4.56 (s, 2H, OCH₂Ph), 7.33 (br s, 5H, OCH₂Ph).

A stirred suspension of 20 mmol 2,4-dihydroxyacetophenone, 20 mmol β-bromoethylbenzylether and 24 mmol K₂CO₃ in 60 mL of acetone was heated to reflux for 16 h. The solvent was evaporated and the residue resuspended in 100 mL H₉O and extracted with CHCl₃ (2×100 mL). The combined layers were dried over sodium sulfate and concentrated under reduced pressure. The crude product was purified by column chromatography (CH₂Cl₂) to give 3.7 g (65%) of a colorless liquid. ¹H NMR (CDCl₃): δ 2.53 (s, 3H, CH₃), 3.81 (t, 2H, J=6 Hz, BnOCH₂CH₂O), 4.14 (t, 2H, J=6 Hz, BnOCH₂CH₂O), 4.60 (s, 2H, OCH₂Ph), 6.40 (m, 2H, C3H+C5H), 7.33 (s, 5H, OCH₂Ph), 7.60 (d, 1H, J=9 Hz, C6H), 12.71 (s, 1H, OH).

3,4-dibenzyloxybenzaldehyde (3) was reacted with 2-hydroxy -4-(2-benzyloxyethoxy)-acetophenone (Id) in a similar way as described for 4b. The brown oil resulting from step 1 was crystallized from ethanol/diethylether to give 2.0 g (49%). In the second step the product was extracted with CHCl3 (2×100 mL). The organic layers were dried over sodiumsulfate and evaporated under reduced pressure. The product was crystallized from methanol/CHCl₃ (4:1) to give 1.2 g (59%) of 4d. ¹H-NMR (CDCl₃): δ 3.87 (t, 2H, J=6 Hz, BnOCH₂CH₂O), 4.25 (t, 2H, J=6 Hz, BnOCH₂CH₂O), 4.64 (s, 2H, PhCH₂-OCH₂CH₂O), 5.24 (s, 4H, C3′OCH₂Ph+C4′OCH₂Ph), 6.88-7.04 (m, 3H, C8H+C5′H+C6H), 7.24-7.51 (m, 15H, PhOCH₂CH₂, C3′OCH₂Ph+C4′OCH₂Ph), 7.78 (dd, 1H, J=9 Hz, 2 Hz, C6′H), 7.85 (d, 1H, J=2 Hz, C2′H), 8.05 (d, 1H, J=9 Hz, C5H). 70 mL 0.8% w/v NaOH i.p.v. 100 mL 5.4% NaOH.

3′,4′-dibenzyloxy-7-methoxyflavone (5b)

2.2 mmol of potassium-tert-butoxide was added to a suspension of 2.0 mmol 4a in 50 mL dry THF, followed by 8.8 mmol CH₃I and the mixture was stirred at room temperature for 16 h. 100 mL H₂O was added and the mixture was extracted with CH₂Cl₂ (2×100 mL). The combined organic layers were dried over sodium sulfate and concentrated under reduced pressure to give 0.9 g (100%) 5b as a white solid. ¹H NMR (CDCl): δ 3.87 (s, 3H, OCH₃), 5.19 (s, 4H, OCH₂Ph×2), 6.55 (s, 1H, C3H), 6.8-7.0 (m, 3H, C5′H+C6H+C8H), 7.26-7.44 (m, 12H, OCH₂Ph×2+C2′H+C6′H), 8.04 (d, 1H, J=9 Hz, C5H).

3′4′-dibenzyloxy-3-methoxyflavone (6b)

The reaction was carried out with 4b, using the same method as described for 5b. The product was crystallized twice from ethyl acetate and hexane (2×) to give 240 mg (47%). ¹H NMR (CDCl₃): δ 3.74 (s, 3H, OCH₃), 5.28 (s, 4H, OCH₂Ph), 6.98 (d, J=7 Hz, C5′H), 7.3-7.7 (m, 14H, OCH₂Ph×2+C6H+C8H+C7H+C6′H), 7.82 (d, 1H, J=2 Hz, C2′H), 8.23 (dd, 1H, J=7 Hz, 2 Hz, C5H).

3-methoxy-7,3′,4′-tribenzyloxyflavone (7b)

4c was reacted as described for 5b. Yield 1.0 g (96%). ¹H NMR (CDCl₃): δ 3.70 (s, 3H, OCH₃), 5.14 (s, 2H, C7OCH₂Ph), 5.25 (s, 4H, C3′OCH₂Ph+C4′OCH₂Ph), 6.90 (d, 1H, J=2 Hz, C8H), 7.0-7.1 (m, 2H, C5′H+C6H), 7.28-7.49 (m, 15H, 3× OCH₂Ph), 7.66 (dd, 1H, J=7 Hz, 2 Hz, C6′H), 7.77 (d, 1H, J=2 Hz, C2′H), 8.12 (d, 1H, J=9 Hz, C5H).

3′,4′-dibenzyloxy-7-hydroxyethoxyflavone (5c)

A mixture of 2.2 mmol 4a and 44 mmol ethylenecarbonate was heated to 95° C., 23 mmol powdered K₂CO₃ was added and the reaction mixture was stirred at 95° C. for 1 h. 50 mL chloroform was added and the K₂CO₃ was filtered. The filtrate was washed with 5 N HCl (3×10 mL), dried over sodium sulfate and concentrated under reduced pressure. The product was recrystallized from ethanol to give 5c as a yellow powder. ¹H NMR (DMSO): δ 3.75 (t, 2H, J=6 Hz, OCH₂CH₂OH), 4.17 (t, 2H, J=6 Hz, OCH₂CH₂OH), 5.23 (s, 2H, C3′OCH₂Ph), 5.29 (s, 2H, C4′OCH₂Ph), 6.92 (s, 1H, C3H), 7.08 (dd, 1H, J=9 Hz, 2 Hz, C5′H), 7.25 (d, 1H, J=7 Hz, C8H), 7.3-7.6 (m, 10H, OCH₂Ph×2), 7.73 (dd, 1H, J=9 Hz, 2 Hz, C6′H), 7.78 (d, 1H, J=2 Hz, C2′H), 7.94 (d, 1H, J=7 Hz, C5H).

3′,4′-dibenzyloxy-3-hydroxyethoxyflavone (6c)

The reaction was carried out with 4b, using the same method as described for 5c: yield 240 mg (49%) of 6c as a light-yellow solid. ¹H NMR (CDCl₃): δ 3.69 (t, 2H, J=6 Hz, CH₂OCH₂Ph), 4.27 (t, 2H, J=6 Hz, C3OCH₂), 4.40 (s, 2H, OCH₂Ph), 5.14 (s, 2H, OCH₂Ph), 5.27 (s, 2H, OCH₂Ph), 6.86 (d, 1H, J=9 Hz, C5′H), 7.2-7.5 (m, 12H, OCH₂Ph×3+C6H+C8H), 7.64 (dt, 1H, J=7 Hz, 2 Hz, C7H), 7.8-7.9 (m, 2H, C2′H+C6′H), 8.20 (dd, J=7 Hz, 2 Hz, C5H).

3-hydroxyethoxy-7,3′,4′-tribenzyloxyflavone (7c)

The reaction was carried out with 4c, using the same method as described for 5c. The product was recrystallized from ethanol and chloroform to give 1.15 g (96%) of 7c as a light-yellow (?@) solid. Mp: 136,7° C. ¹H NMR (CDCl₃): δ 3.6-3.8 (m, 4H, OCH₂CH₂O), 5.15 (s, 2H, OCH₂Ph), 5.25 (s, 2H, OCH₂Ph), 5.26 (s, 2H, OCH₂Ph), 6.92 (d, 1H, J=2 Hz, C8H), 7.0-7.1 (m, 2H, C6H+C5′H), 7.2-7.5 (m, 10H, OCH₂Ph×2), 7.63 (dd, 1H, J=8 Hz, 2 Hz, C6′H), 7.74 (d, 1H, J=2 Hz, C2′H), 8.12 (d, 1H, J=9 Hz, C5H).

3-(3′,4′-dibenzyloxyflavon-7-yl)-propyltrimethylammoniumchloride (5d)

To a solution of 6.0 mmol 4a in 30 mL DMSO was added 17.4 mmol K₂CO₃ and 1.2 mL (12.0 mmol) 1-bromo-3-chloropropane and the mixture was stirred at room temperature for 5 h. The DMSO was evaporated under reduced pressure. The residue was suspended in 100 mL CH₂Cl₂, filtered and concentrated in vacuo to give 3.0 gram (95%) 7-(3-chloropropoxy)-3′,4′-dibenzyloxyflavone; mp 152.6-153.4° C. ¹H NMR (CDCl₃): δ 2.29 (m, 2H, CCH₂C), 3.76 (t, 2H, J=6 Hz, CH₂Cl), 4.22 (t, 2H, J=6 Hz, OCH₂), 5.23 (s, 4H, OCH₂Ph×2), 6.59 (s, 1H, C3H), 6.89-7.02 (m, 3H, C6H+C8H+C5′H), 7.30-7.50 (m, 12H, OCH₂Ph×2+C2′H+C6′H), 8.08 (d, 1H, J=9 Hz, C5H).

A mixture of 2.5 mmol 7-(3-chloropropoxy)-3′,4′-dibenzyloxyflavone in 15 mL dioxane, 15 mL trimethylamine (33% in ethanol) and 2 mmol K₂CO₃ was heated to 100° C. for 56 h in a stainless steel bomb. The mixture was filtered and the solvents were evaporated under reduced pressure. The product was recrystallized twice from isopropanol/ethanol to give 1.26 g (86%) 5d as a white solid. ¹H NMR (DMSO): δ 2.27 (m, 2H, CCH₂C), 3.12 (s, 9H, N(CH₃)₃), 3.82 (t, 2H, J=6 Hz, CH₂N), 4.24 (t, 2H, J=6 Hz, OCH₂), 5.24 (s, 2H, C3′OCH₂Ph), 5.27 (s, 2H, C4′OCH₂Ph), 6.93 (s, 1H, C3H), 7.0-8.0 (m, 16H, C6′H+C2′H+C6H+C8H+C5′H+C3′OCH₂Ph+C4′OCH₂Ph+C5H).

3-(3′4′-dibenzyloxyflavon-3-yl)-propyltrimethylammoniumbromide (6f)

4.5 mL (44.3 mmol) 1,3-dibromopropane was added to a solution of 11.1 mmol 4b and 11.8 mmol potassium-tert-butoxide in 250 mL dry THF. The reaction mixture was stirred at room temperature for 19 h and subsequently heated to reflux under an atmosphere of dry nitrogen for 5 h. The mixture was dissolved in 250 mL CH₂Cl₂ and washed with HO. The organic layer was dried over magnesium sulfate and evaporated under reduced pressure. The crude product was purified by column chromatography (ethyl acetate/hexane 1:1 (v/v)) and recrystallized from ethylacetate and hexane to give 2.71 g (42%) 3-(3-bromopropoxy)-3′,4′-dibenzyloxyflavone. ¹H NMR (CDCl₃): δ 218 (m, 2H, CCH₂C), 3.47 (t, 2H, J=6 Hz, CH₂Cl), 4.13 (t, 2H, J=6 Hz, OCH₂), 5.23 (s, 2H, OCH₂Ph), 5.27 (s, 2H, OCH₂Ph), 7.04 (s, 1H, C3H), 7.25-7.5 (m, 3H, C6H+C8H+C5′H), 7.6-7.8 (m, 12H, OCH₂Ph×2+C2′H+C6′H), 8.23 (dd, 1H, J=7 Hz, 2 Hz, C5H).

A mixture of 4.7 mmol 3-(3-bromopropoxy)-3′,4′-dibenzyloxyflavone in 15 mL dioxane and 25 mL trimethylamine (33% in ethanol) was heated to 100° C. for 17 h in a stainless steel bomb. The solvents were evaporated under reduced pressure and the product was recrystallized from ethanol and hexane to give 2.1 g (71%) 6f as an off-white powder. ¹H NMR (DMSO): δ 2.03-02.18 (m, 2H, CH₂), 3.38 (s, 9H, N(CH₃)₃), 3.45-3.55 (m, 2H, CH₂N), 4.0 (t, 2H, J=7 Hz, OCH₂), 5.24 (s, 2H, OCH₂Ph), 5.28 (s, 2H, OCH₂Ph), 7.26-7.55 (m, 11H, 2× OCH₂Ph+C5′H), 7.70-7.92 (m, 5H, C6H+C8H+C7H+C6′H+C2′H), 8.10 (d, 1H, J=9 Hz, C5H).

6-(3′4′-dibenzyloxyflavon-3-yl)-hexyltrimethylammoniumchloride (6g)

4b was alkylated using 1-chloro-6-iodohexane, employing the same method as described for 5d. The crude product was purified by column chromatography (CH₂Cl₂) to give 1.6 g (94%) 3-(6-chlorohexyloxy)-3′,4′-dibenzyloxyflavone. ¹H NMR (CDCl₃): δ 1.36 (m, 4H, CH₂), 1.68 (m, 4H, CH₂), 3.47 (t, 2H, J=6 Hz, CH₂Cl), 3.99 (t, 2H, J=6 Hz, OCH₂), 5.23 (s, 2H, OCH₂Ph), 5.27 (s, 2H, OCH₂Ph), 7.03 (d, 1H, J=8 Hz, C5′H), 7.25-7.5 (m, 12H, 2× OCH₂Ph+C6H, C8H), 7.66 (dt, 1H, J=7 Hz, 2 Hz, C7H), 7.71 (dd, 1H, J=8 Hz, 1 Hz, C6′H), 7.80 (d, 1H, J=1 Hz, C2′H), 8.23 (dd, 1H, J=7 Hz, 2 Hz, CSH).

3-(6-chlorohexyloxy)-3′,4′-dibenzyloxyflavone was reacted in a similar way as described for 5d, without the addition of K₂CO₃. The product was crystallized from ethanol and ether to give 1.14 g (69%) 6g. ¹H NMR (CDCl₃): δ 1.2-1.7 (m, 8H, CH₂), 3.28 (s, 9H, N⁺(CH₃)₃), 3.42 (t, 2H, J=7 Hz, CH₂N), 3.86 (t, 2H, J=7 Hz, OCH₂), 5.19 (s, 2H, OCH₂Ph), 5.19 (s, 2H, OCH₂Ph), 7.03 (d, 1H, J=9 Hz, C5′H), 7.10-7.70 (m, 15H, 2× OCH₂Ph+C6H+C8H+C7H+C6′H+C2′H), 8.13 (dd, 1H, J=9 Hz, C5H).

8-(3′4′-dibenzyloxyflavon-3-yl)-octyltrimethylammoniumbromide (6 h)

4b was alkylated using 1,8-dibromooctane, employing the same method as described for 5d. Yield: 1.05 g (82%) 3-(8-bromooctyloxy)-3′,4′-dibenzyloxyflavone. ¹H NMR (CDCl₃): δ 1.2-1.5 (m, 8H, O(CH₂)₃(CH₂)₄CH₂Br), 1.5-1.9 (m, 4H, OCH₂(CH₂)₂(CH₂)₅Br), 3.34 (t, 2H, J=7 Hz O(CH₂)₇CH₂Br), 3.97 (t, 2H, J=7 Hz, OCH₂(CH₂)₇Br), 5.22 (s, 2H, C3′OCH₂Ph), 5.25 (s, 2H, C4′OCH₂Ph), 7.02 (d, 1H, J=9 Hz, C5′H), 7.25-7.55 (m, 12H, C3′OCH₂Ph+C4′OCH₂Ph+C6H+C8H), 7.59-7.73 (m, 2H, C6′H+C7H), 7.81 (d, 1H, J=2 Hz, C2′H), 8.22 (dd, 1H, J=8.0 Hz, 2 Hz, C5′H).

3-(8-bromooctyloxy)-3′,4′-dibenzyloxyflavone was reacted in a similar way as described for 6f, with the modification that 3-(8-bromooctyloxy)-3′,4′-dibenzyloxyflavone was dissolved in ethanol and 1,4-dioxane. Yield: 0.93 g (91%) of a yellow solid. ¹H NMR (CDCl₃): δ 1.2-1.7 (m, 12H, CH₂), 3.23 (s, 9H, N⁺(CH₃)₃), 3.38 (t, 2H, J=9 Hz, O(CH₂)₇CH₂N⁺(CH₃)₃), 3.97 (t, 2H, J=6 Hz, OCH₂), 5.18 (s, 2H, C3′OCH₂Ph), 5.21 (s, 2H, C4′OCH₂Ph), 7.07 (d, 1H, J=9 Hz, C5′H), 7.33-7.50 (m, 12H, C3′OCH₂Ph+C4′OCH₂Ph+C6H+C8H), 7.61-7.72 (m, 2H, C6′H+C7H), 7.79 (d, 1H, J=2 Hz, C2′H), 8.18 (dd, 1H, J=7 Hz, 2 Hz, C5H).

3-(7,3′,4′-tribenzyloxyflavon-3-yl)-propyltrimethylammoniumchloride (7d)

4c was alkylated using 1-bromo-3-chloropropane, employing the same method as described for 5d. The product was purified by column chromatography (CH₂Cl₂) to give 3.2 g (84%) 3-(3-chloropropoxy)-7,3′,4′-tribenzyloxyflavone as a solid; mp: 136.1-137.3° C. ¹H NMR (CDCl₃): δ 2.06 (m, 2H, OCH₂CH₂CH₂Cl), 3.59 (t, 2H, J=7 Hz, OCH₂CH₂CH₂Cl), 4.08 (t, 2H, J=7 Hz, OCH₂CH₂CH₂Cl), 5.15 (s, 2H, C7OCH₂Ph), 5.22 (s, 2H, C3′OCH₂Ph), 5.24 (s, 2H, C4′OCH₂Ph), 6.89 (d, 1H, J=1 Hz, C8H), 7.0-7.1 (m, 2H, C5′H+C6H), 7.3-7.8 (m, 17H, 3× OCH₂Ph+C2′H+C6′H), 8.12 (d, 1H, J=7 Hz, C5H).

A mixture of 2.0 mmol 3-(3-chloropropoxy)-7,3′,4′-tribenzyloxyflavone in 15 mL trimethylamine (33% in ethanol) was heated to 100° C. for 64 h in a stainless steel bomb. The solvents were evaporated under reduced pressure and the product was crystallized from toluene to give 167 mg (12%) of a white solid. Mp: 174.7-174.8° C. ¹H NMR (DMSO): δ 2.08 (m, 2H, OCH₂CH₂CH₂N(CH₃)₃), 3.06 (s, 9H, N⁺(CH₃)₃), 3.52 (m, 2H, OCH₂CH₂CH₂N (CH₃)₃), 4.08 (t, 2H, J=7 Hz, OCH₂CH₂CH₂N(CH₃)₃), 5.23 (s, 2H, C7OCH₂Ph), 5.27 (s, 4H, 2× OCH₂Ph), 7.14 (dd, 1H, J=9 Hz, 2 Hz, C5′H), 7.20-7.70 (m, 19H, 3× OCH₂Ph+C6H+C8H+C6′H+C2′H), 7.98 (d, 1H, J=9 Hz, C5H).

3′,4′-dibenzyloxy-7-(3-dimethylaminopropoxy)flavone (5e)

4a was reacted in a similar way as described for 5d using dimethylamine (33% in ethanol). The residue was suspended in acidic water (pH=1-2), washed with chloroform (2×100 mL), basified with 1 M NaOH and extracted with chloroform (2×150 mL). The organic layers were dried over sodium sulfate and concentrated in vacuo to give 0.8 g (60%) 5e. ¹H NMR (CDCl₃+20% DMSO): δ 2.37 (m, 2H, CCH₂C), 2.86 (d, 6H, J=7 Hz, N(CH₃)₂), 3.30 (m, 2H, CH₂N), 4.30 (t, 2H, J=6 Hz, OCH₂), 5.21 (s, 2H, C3′OCH₂Ph), 5.23 (s, 2H, C4′OCH₂Ph), 6.70 (s, 1H, C3H), 6.98-7.13 (m, 3H, C6H+C8H+C5′H), 7.33-7.56 (m, 12H, OCH₂Ph×2+C2′H+C6′H), 7.98 (d, 1H, J=9 Hz, C5H).

3′,4′-dibenzyloxy-3-(3-dimethylaminopropoxy)flavone (6e)

4b was alkylated as described for 5d. After evaporation of the DMSO the residue was dissolved in 100 mL H₂O and extracted with chloroform (2×100 mL). The organic layers were dried over sodium sulfate and evaporated under reduced pressure to give 1.5 g (95%) 3-((3-chloropropoxy)-3′,4′-dibenzyloxyflavon. 3H NMR (CDCl3): δ 2.08 (m, 2H, OCH₂CH₂CH₂Cl), 3.60 (t, 2H, J=7 Hz, OCH₂CH₂CH₂Cl), 4.11 (t, 2H, J=7 Hz, OCH₂CH₂CH₂Cl), 5.26 (s, 4H, C3′OCH₂Ph+C4′OCH₂Ph), 7.12 (d, 1H, J=9 Hz, C5′H), 7.26-7.52 (m, 17H, 3× OCH₂Ph+C6H+C8H), 7.60-7.70 (m, 3H, C2′H+C6′H+C7H), 8.20 (d, 1H, J=9 Hz, C5H).

A mixture of 2.0 mmol 3-(3-chloropropoxy)-3′,4′-dibenzyloxyflavon in 10 mL ethanol and 10 mL dioxane, 2.0 mmol NaI, 4.0 mmol Na₂CO₃ and 25 mL dimethylamine (33% in ethanol) was heated to 60° C. for 56 h in a stainless steel bomb. The solvents were evaporated under reduced pressure. The resulting brown oil was directly used for further reaction.

(3′4′-dibenzyloxyflavon-3-oxy)acetic Acid (6d)

To a stirred solution of 2.0 mmol 4b and 4.0 mmol potassium-tert-butoxide in 25 mL dry THF was added 6.1 mmol ethyl chloroacetate. The reaction mixture was stirred at room temperature for 16 h. After acidification with concentrated HCl, 100 mL ice-cold H O was added and the mixture was extracted with CH₂Cl₂ (4×25 mL) The organic layers were dried over magnesium sulfate and evaporated in vacuo. The product was crystallized from ethylacetate to give 0.62 g (1.15 mmol 57%) of ethyl (3′,4′-dibenzyloxyflavon-3-oxy)acetate as an off-white powder.

The powder was dissolved in 1,4-dioxane and a solution of 2.07 mmol NaOH dissolved in 7.5 mL methanol was added. The reaction mixture was stirred at room temperature for 16 h. After acidification with glacial acetic acid and the addition of 20 mL H₂O, the mixture was extracted with CH₂Cl₂ (2×20 mL). The organic layers were dried over sodium sulfate and evaporated under reduced pressure. The resulting yellow powder consisted of the desired product and 1,4-dioxane and was used without further purification. ¹H NMR (CDCl₃): δ 4.0 (s, 2H, OCH₂COOH), 5.21 (s, 2H, C3′OCH₂Ph), 5,23 (s, 2H, C4′OCH₂Ph), 7.00 (d, 1H, J=9 Hz, C5′H), 7.18-7.74 (m, 15H, 2× OCH₂Ph+C2′H+C6′H+C6H+C7H+C8H), 8.2 (dd, 1H, J=8.0 Hz, 1 Hz, C5H).

3′1,4′-dibenzyloxy-3-tetraacetylglucoseflavone (6i)

To a solution of 1 mmol 4b and 1.5 mmol acetobromoglucose in 10 mL dry pyridine (+molecular sieves 5 Å) 2.2 mmol silveroxide was added. After stirring at room temperature for 1 h silver salts were removed by filtration over celite and silica. The solvent was concentrated in vacuo and the crude product was purified by column chromatography to give 700 mg (90%) of a greenish ‘glass’. ¹H NMR (CDCl₃): δ 1.78 (s, 3H, Ac), 1.98 (s, 3H, Ac), 1.99 (s, 3H, Ac), 2.07 (s, 3H, Ac), 3.58 (dt, 1H, J=10 Hz, 1 Hz, CH—O), 3.9-4.1 (m, 2H, CH₂OAC), 5.0-5.3 (m, 3H, 3× CH—O), 5.24 (s, 2H, OCH₂Ph), 5.28 (s, 2H, OCH₂Ph), 5.68 (d, 1H, J=8 Hz, acetal-H), 6.99 (d, 1H, J=9 Hz, C5′H), 7.3-7.8 (m, 15H, 2× OCH₂Ph+C2′H+C6′H+C6H+C7H+C8H), 8.18 (dd, 1H, J=8 Hz, 2 Hz, C5H).

3′,4′-dibenzyloxy-3-hexaacetylrutinoseflavone (6j)

A suspension of 5 g rutine in 150 mL 50% acetic acid was heated to reflux under an atmosphere of dry nitrogen for 6 h. 100 mL H₂O was added and the reaction mixture was kept overnight at 4° C. to form a precipitate. After filtration the filtrate was concentrated in vacuo. After addition of 15 mL pyridine and 15 mL acetic acid anhydride the reaction mixture was stirred for 3 h at room temperature. CH₂Cl₂ was added and the organic layer was washed with H₂O(2×), dried over sodium sulfate and evaporated to dryness. The product was purified by column chromatography (ether) to give 1.28 g (27%) β-heptaacetylrutinose as a white ‘glas’.

To a solution of 2.1 mmol β-heptaacetylrutinose in 12 mL dry CH₂Cl₂ 4.9 mmol TiBr₄ in 12 mL dry CH₂Cl₂ was added and the reaction mixture was stirred for 17 h at room temperature under an atmosphere of dry nitrogen. The solution was neutralized with NaAc, filtered over a glassfilter and subsequently over celite and evaporated under reduced pressure. The resulting orange/brown oil (1.6 g) containing α-acetobromorutinose was used without further purification.

4b was reacted with a-acetobromorutinose as described for 6i to give 634 mg (46%) 6j as a white-greenish ‘glass’. ¹H NMR (CDCl₃): δ 0.97 (d, 3H, J=6 Hz, CH₃), 1.86 (s, 3H, Ac), 1.88 (s, 3H, Ac), 1.96 (s, 3H, Ac), 1.97 (s, 3H, Ac), 2.00 (s, 3H, Ac), 2.03 (s, 3H, Ac), 3.3-3.7 (m, 4H, 2× CH—O+CH₂O), 4.49 (s, 1H, acetal-H), 4.8-5.3 (m, 6H, CH—O), 5.21 (s, 2H, OCH₂Ph), 5.23 (s, 2H, OCH₂Ph), 5.65 (d, 1H, J=8 Hz, acetal-H), 7.03 (d, 1H, J=9 Hz, C5′H), 7.2-7.7 (m, 15H, 2× OCH₂Ph+C2′H+C6′H+C6H+C7H+C8H), 8.15 (d, J=8 Hz, C5H).

General Procedure for Debenzylation (Method A)

A suspension of 2 mmol of the required compound in 50 mL HCl (36%) and 50 mL glacial acid (99%) was heated to 100° C. under an atmosphere of dry nitrogen for 2-3 h. The solvents were evaporated under reduced pressure and the product was crystallized.

3′,4′,7-trihydroxyflavone (8a)

Crystallized yield: 82%; ¹H NMR (CDCl₃+20% DMSO): δ 6.35 (br s, OH+H₂O), 6.59 (s, 1H, C3H), 6.8-6.9 (m, 3H, C8H, C6H, C5′H), 7.26 (d, 1H, J=9 Hz, C6′H), 7.34 (s, 1H, C2′H), 7.86 (d, 1H, J=8 Hz, C5H).

3′,4′-dihydroxy-7-methoxyflavone (8b)

Crystallized from ethanol/chloroform 4:1 to give 0.5 g (88%) yellow powder. Mp>240° C. ¹H NMR (CDCl₃+20% DMSO): δ 3.87 (s, 3H, OCH₃), 6.50 (s, 1H, C—OH), 6.8-7.0 (m, 3H, C5′H+C6H+C8H), 7.2-7.4 (m, 2H, C6′H+C2′H), 7.93 (d, 1H, J=9 Hz, C5H)

3′,4′-dihydroxy-7-hydroxyethoxyflavone (8c)

¹H NMR (DMSO): δ 3.76 (t, 2H, J=5 Hz, CH₂OH), 4.13 (t, 2H, J=5 Hz, C7OCH₂), 6.66 (s, 1H, C3H), 6.90 (d, 1H, J=8 Hz, C5′H), 7.07 (d, 1H, J=7 Hz, C6H), 7.28 (s, 1H, C8H), 7.35-7.50 (m, 2H, C2′H+C6′H), 7.89 (d, 1H, J=8 Hz, C5H).

3′4′-dihydroxy-7-(3-trimethylaminopropoxy)flavone (8d)

Crystallized from ethanol/diethylether yielding 0.8 g (91%) 3d′, mp>210° C. ¹H NMR (DMSO+D2O): δ 2.22 (m, 2H, (CH₃)₃NCH₂CH₂CH₂O), 3.12 (s, 9H, (CH₃)₃NCH₂CH₂CH₂O), 3.50 (m, 2H, (CH₃)₃NCH₂CH₂CH₂O), 3.89 (m, 2H, (CH₃)₃NCH₂CH₂CH₂O), 6.26 (s, 1H, C3H), 6.55-7.2 (m, 5H, C5′H+C6′H+C8H+C5H+C6H), 7.58 (d, 1H, J=9 Hz, C5H).

3′,4′-dihydroxy-7-(3-dimethylaminopropoxy)flavone (8e)

The residue was dissolved in CH₂Cl₂. The resulting suspension was filtered and the residue gave 0.5 g (93%) 3e′. Mp 173.6° C. ¹H NMR (D₂O): δ 2.02 (m, 2H, CH₂) 2.88 (s, 6H, N(CH₃)₂), 3.18 (t, 2H, J=7 Hz, CH₂N), 3.60 (m, 2H, C7OCH₂), 5.70 (s, 1H, C3H), 6.10 (s, 1H, C8H), 6.30-6.60 (m, 4H, C6H+C2′H+C5′H+C6′H), 7.13 (d, 1H, J=9 Hz, C5H).

3′3,3′,4′-trihydroxyflavone (9a)

Crystallized yield: 69%; ¹H NMR (DMSO): δ 6.89 (d, 1H, J=9 Hz, C5′H), 7.44 (t, 1H, J=8 Hz, C6H), 7.61 (dd, 1H, J=9 Hz, 2 Hz, C6′H), 7.65-7.75 (m, C7H+C8H+C2′H), 8.09 (d, 1H, J=8 Hz, C5H), 9.4 (br s, 3H, OH).

3′4′-dihydroxy-3-(2-ethoxycarboxylic acid)flavone (9d)

Crystallized yield: 0.21 g (43%) 6c′ as a green powder; ¹H NMR (DMSO): δ 4.74 (s, 2H, OCH₂COOH), 6,89 (d, 1H, J=9 Hz, C5′H), 7.48 (t, 1H, J=7.4 Hz, C6H), 7.58-7.86 (m, 4H, C6′H+C7H+C8H+C2′H), 8.09 (dd, 1H, J=8 Hz, 2 Hz, C5H), 9.32 (s, 1H, C3′OH), 9.79 (s, 1H, C4′OH), 12.92 (br s, 1H, CH₂COOH).

3′4′-dihydroxy-3-(3-trimethylaminopropoxy)flavone (9f)

Crystallized yield: 91%; ¹H NMR (DO): δ 1.86 (m, 2H, CH₂), 2.98 (s, 9H, N(CH₃)₃), 3.12 (m, 2H, CH₂N), 3.45 (t, 2H, J=6 Hz, OCH₂), 6.49 (d, 1H, J=9 Hz, C5′H), 6.74 (d, 1H, J=8 Hz, C8H), 6.8-6.9 (m, 2H, C2′H+C6′H), 7.10 (t, 1H, J=8 Hz, C6H), 7.37 (t, 1H, J=8 Hz, C7H), 7.46 (d, 1H, J=8 Hz, C5H).

3′4′-dihydroxy-3-(6-trimethylaminohexyloxy)flavone (9g)

Crystallized yield: 51%; ¹H NMR (DMSO): δ 1.15-1.8 (m, 8H, CH₂), 3.04 (s, 9H, N⁺(CH₃)₃), 3.36 (m, 2H, CH₂N), 3.98 (t, 2H, J=6 Hz, OCH₂), 6.96 (d, 1H, J=8 Hz, C5′H), 7.4-7.9 (m, 5H, C8H+C2′H+C6′H+C6H+C7H), 8.08 (dd, 1H, J=8 Hz, 1 Hz, C5H), 9.44 (s, 1H, OH), 9.91 (s, 1H, OH).

3′4′-dihydroxy-3-(8-trimethylaminooctyloxy)flavone (9 h)

Crystallized yield: 54%; ¹H NMR (DMSO): δ 1.15-1.75 (m, 12H, CH₂), 3.04 (s, 9H, N⁺(CH₃)₃), 3.28 (m, 2H, CH₂N), 3.97 (t, 2H, J=6 Hz, OCH₂), 6.91 (d, 1H, J=8 Hz, C5′H), 7.4-7.9 (m, 5H, C8H+C2′H+C6′H+C6H+C7H), 8.07 (dd, 1H, J=8 Hz, 1 Hz, C5H), 9.44 (br s, 1H, OH), 9.67 (br s, 1H, OH)

3-methoxy-7,3′,4′-trihydroxyflavone (10b)

Crystallized from ethanol/chloroform (4:1) to give 0.5 g (90%) yellow powder. Mp: 281.4° C. ¹H NMR (DMSO): δ 3.76 (s, 3H, OCH₃), 6.8-7.0 (m, 3H, C5′H+C6H+C8H), 7.42 (dd, 1H, J=8 Hz, 1 Hz, C6′H), 7.55 (d, 1H, J=1 Hz, C2′H), 7.86 (d, 1H, J=8 Hz, C5H), 9.90 (br s, 1H, C7OH)

3-hydroxyethoxy-7,3′,4′-trihydroxyflavone (10c)

Washed with CHCl₃ to give 0.5 g (80%) orange powder. Mp: >300° C. ¹H NMR (DMSO): δ 4.66 (m, 2H, OCH₂CH₂OH), 5.00 (m, 2H, OCH₂CH₂OH), 7.05 (d, 1H, J=8 Hz, C5′H), 7.35 (d, 1H, J=8 Hz, C6H), 7.46 (s, 1H, C8H), 7.88-8.00 (m, 2H, C6′H+C2′H), 8.06 (d, 1H, J=9 Hz, C5H), 9.88 (br s, 1H, C7OH), 10,85 (br s, 1H, C3′OH), 12.35 (br s, 1H, C4′OH).

3-(7,3′,4′-trihydroxyflavon-3-yl)-propyltrimethylammoniumchloride (10d)

Crystallized from methanol/ether (1:1) to give 95 mg (95%) brown-white powder. Mp: decomposes>200° C. ¹H NMR (DMSO): δ 2.11 (m, 2H, CH₂), 3.07 (s, 9H, N⁺(CH₃)₃), 3.48 (m, 2H, CH₂N), 3.97 (t, 2H, J=6 Hz, OCH₂), 6.9-7.0 (m, 3H, C5′H+C6H+C8H), 7.43 (dd, 1H, J=8 Hz, 2 Hz, C6′H), 7.54 (s, 1H, J=2 Hz, C2′H), 7.88 (d, 1H, J=9 Hz, C5H), 9.56 (s, 1H, OH), 9.76 (s, 1H, OH), 10.93 (s, 1H, OH)

General Procedure for Debenzylation (Method B)

To a suspension/solution of 1 mmol of the required compound in methanol 150 mg 10% Pd/C was added. The reaction mixture was stirred at room temperature for 30 min. to 1 h under an atmosphere of 1.5 atm H₂ (g) after replacement of the air by nitrogen. The Pd/C was filtered and the solvent was evaporated under reduced pressure.

3′4′-dihydroxy-3-methoxyflavone (9b)

¹H NMR (DMSO): δ 3.83 (s, 3H, OCH), 6.95 (d, 1H, J=9 Hz, C5′H), 7.45-7.88 (m, 5H, C7H+C6H+C8H+C6′H+C2′H), 8.1 (d, 1H, J=7 Hz, C5H).

3′,4′-dihydroxy-3-hydroxyethoxyflavone (9c)

¹H NMR (DMSO): δ 3.65 (t, 2H, OCH₂CH₂OH), 4.05 (t, 2H, OCH₂CH₂OH), 6.9 (d, 1H, J=9 Hz, C5′H), 7.45 (dt, 1H, J=7 Hz, 1 Hz, C6H), 7.6-7.85 (m, 4H, C7H+C8H+C6′H+C2′H) 8.08 (dd, 1H, J=7 Hz, 1 Hz, C5H).

3′,4′-dihydroxy-3-(3-dimethylaminopropoxy)flavone (9e)

¹H NMR (CDCl₃+20% DMSO): δ 2.11 (t, 2H, OCH₂CH₂CH₂N(CH₃)₂), 2.74 (s, 6H, N(CH₃)₂), 3.24 (m, 2H, CH₂N), 3.85 (t, 2H, J=6 Hz, OCH₂CH₂CH₂N(CH₃)₂), 6.79 (d, 1H, J=8 Hz, C5′H), 7.2-7.6 (m, 4H, C6H+C8H+C6′H+C7H), 7.70 (d, 1H, J=2 Hz, C2′H), 7.95 (dd, 1H, J=8 Hz, 1 Hz, C5H), 8.56 (s, 1H, OH), 8.89 (s, 1H, OH), 11.35 (br s, 1H, N⁺H).

3′,4′-dihydroxy-3-glucoseflavone (9i)

Before debenzylation, the acetyl groups were removed. Yield: 89%; ¹H NMR (DMSO): δ 3.0-3.7 (m, 6H, 4× CH+1× CH₂ sugar), 5.55 (m, 1H, acetal-H), 6.86 (d, 1H, J=9 Hz, C5′H), 7.48 (t, 1H, J=7 Hz, C6H), 7.6-7.9 (m, 4H, C2′H+C6′H+C7H+C8H), 8.08 (d, 1H, J=8 Hz, C5H). ¹³C NMR (DMSO): 60.7 (CH₂), 69.6 (CH), 73.7 (CH), 76.3 (CH), 77.3 (CH), 100.5 (acetal-CH), 114.9 (CH), 116.2 (CH), 117.9 (CH), 121.2 (C), 121.4 (CH), 123.0 (C), 124.7 (2× CH), 133.6 (CH), 135.4 (C), 144.6 (C), 148.1 (C), 154.2 (C), 155.7 (C), 173.1 (CO).

3′,4′-dihydroxy-3-rutinoseflavone (9j)

Before debenzylation, the acetyl groups were removed. Yield: 95%; ¹H NMR (DMSO): δ 0.97 (d, 3H, J=6 Hz, CH₃ 3.0-3.7 (m, 10H, 8× CH+1× CH₂ sugar), 5.46 (m, 2H, 2× acetal-H), 6.86 (d, 1H, J=9 Hz, C5′H), 7.46 (t, 1H, J=7 Hz, C6H), 7.6-7.9 (m, 4H, C2′H+C6′H+C7H+C8H), 8.08 (d, 1H, J=8 Hz, C5H). ¹³C NMR (DMSO): δ 17.5 (CH₃) 66.6 (CH₂), 68.0 (CH), 69.8 (CH), 70.1 (CH), 70.2 (CH), 71.6 (CH), 73.7 (CH), 75.7 (CH), 76.4 (CH), 100.4 (acetal-CH), 100.8 (acetal-CH), 115.0 (CH), 116.2 (CH), 117.9 (CH), 121.2 (C), 121.5 (CH), 122.9 (C), 124.7 (2× CH), 133.6 (CH), 135.4 (C), 144.5 (C), 148.0 (C), 154.3 (C), 156.0 (C), 173.2 (CO).

7-(2-hydroxyoxyethoxy)-3,3′,4′-trihydroxyflavone (11a)

Crystallized yield: 240 mg (92%); ¹H NMR (DMSO): δ 3.77 (q, 2H, J=6 Hz, HOCH₂CH₂O), 4.15 (t, 2H, J=6 Hz, HOCH₂CH₂O), 4.99 (t, 1H, J=6 Hz, HOCH₂CH₂O), 6.89 (d, 1H, J=9, C5′H), 7.05 (dd, 1H, J=9 Hz, 2 Hz, C6H), 7.22 (d, 1H, J=2 Hz, C8H), 7.55 (dd, 1H, J=8 Hz, 2 Hz, C6′H), 7.74 (d, 1H, J=2 Hz, C2′H), 7.97 (d, 1H, J=9 Hz, C5H), 9.17 (1H, 9, C3OH), 9.27 (s, 1H, C3′OH), 9.57 (s, 1H, C4′OH).

Experiment 1: Lipid Peroxidation Model

Preparation of hepatic microsomes.

Male Wistar rats (200-220 g) obtained from Harlan Nederland B.V. (Horst, The Netherlands) were sacrificed by decapitation. Livers were removed and homogenized in ice-cold phosphate buffer (50 mM+0.1 mM EDTA, pH=7.4, 1:2 w/v). The homogenate was centrifuged at 10 000× g (20 min at 4° C.). Subsequently, the supernatant was centrifuged at 100 000× g for 60 min (4° C.). The pellet was resuspended in phosphate buffer and centrifuged again at 100 000× g (60 min, 4° C.). Finally the microsomal pellet was resuspended in phosphate buffer (2 g liver/ml) and 1 mL aliquots were stored at −80° C.

Microsomes were thawed and resuspended in ice-cold Tris-HCl buffer (50 mM, pH=7.4 at 37° C.) and washed by centrifugation. The pellet was resuspended in 2 mL 50 mM Tris-HCl buffer and heated to 100° C. for at least 2 min to remove all enzymatic factors just before resuspending and diluting it in ice-cold Tris buffer to 1.5 mg/mL.

Stock solutions of the flavonoids were freshly prepared in nitrogen-purged DMSO and nanopure water (1:1) just before use. The reaction was started by adding 10 uM freshly prepared FeSO₄ in nitrogen-purged nanopure water. LPO was assayed by measuring thiobarbituric acid (TBA) reactive substances. At t=0, 5, 10, 15, 30, 45 and 60 min an 0.3 mL aliquot of the incubation was mixed with 2 mL of an ice-cold TBA-trichloroacetic acid-HCl-butylhydroxytoluene (BHT) solution to stop the reaction. After heating and centrifugation the absorbance at 535 vs 600 nm was determined. The TBA-trichloroacetic acid-HCl-butylhydroxytoluene solution was prepared by dissolving 41.6 mg TBA/10 ml trichloroacetic acid (16.8% w/v in 0.125 N HCl). To 10 ml TBA-trichloroacetic acid-HCl 1 ml BHT (1.5 mg/ml ethanol) was added.

The IC₅₀ was determined by measuring the % LPO inhibition at several concentrations and interpolating the 50% inhibition point.

Experiment 2: TEAC Model

A solution of 2,2′-azino-bis(3-ethylbenzthiazoline-6-sulfonic acid) (1.23 mg/mL) and azobisamidinepropane (3.96 mg/mL) in 50 mM phosphate buffer (pH=7.4) was heated at 70° C. for 20 min. The amount of ABTS radicals was measured by determination of the absorbance at 734 nm.

Stock solutions of the flavonoids were freshly prepared in nitrogen-purged DMSO and nanopure water (1:1) just before use. A calibration curve of Trolox ((+/−)-6-hydroxy-2,5,7,8-tetramethyl-chroman-2-carboxylic acid) was made in a concentration range from 0 to 20 μM. A mixture of 50 μL test compound and 950 μL ABTS⁻⁺ solution was incubated at 37° C. for 5 min. Subsequently the absorbance at 734 nm was determined on a UV/VIS Spectrophotometer.

The antioxidant-induced decrease in absorbance is directly related to the antioxidant capacity of the compound (solution) being tested.

The Trolox equivalent antioxidant capacity (TEAC) is defined as the concentration (mM) of Trolox having an antioxidant capacity equivalent to 1 mM of the test compound.

TABLE 1
Antioxidant activities in the lipid peroxidation assay (mean ± s.e.m., n = 3-5)
and the TEAC assay (mean ± S.D., n = 3-5).

| Compound | Substituents | | | | | | LPO IC₅₀ | TEAC |
|---|---|---|---|---|---|---|---|---|
| no. | R1(3) | R4(5) | R2(7) | R5(3′) | R6(4′) | R7(5′) | (μM) | (mM) |
| 8a | H | H | OH | OH | OH | H | 11.0 ± 1.3 | 4.3 ± 0.2 |
| 8b | H | H | OCH₃ | OH | OH | H | 2.5 ± 0.8 | 3.3 ± 0.2 |
| 8c | H | H | OCH₂CH₂OH | OH | OH | H | 16.4 ± 1.7 | 4.3 ± 0.7 |
| 8d | H | H | O(CH₂)₃N⁴(CH₃)₃ | OH | OH | H | 37.3 ± 2.4 | 1.9 ± 0.3 |
| 8e | H | H | O(CH₂)₃N(CH₃)₂ | OH | OH | H | 8.1 ± 1.1 | 2.9 ± 0.4 |
| 9a | OH | H | H | OH | OH | H | 1.8 ± 1.0 | 4.5 ± 0.5 |
| 9b | OCH₃ | H | H | OH | OH | H | 1.3 ± 0.7 | 4.4 ± 0.3 |
| 9c | OCH₂CH₂OH | H | H | OH | OH | H | 0.6 ± 0.1 | 4.4 ± 0.4 |
| 9d | OCH₂COOH | H | H | OH | OH | H | 6.5 ± 1.0 | 7.6 ± 0.6 |
| 9e | O(CH₂)₃N(CH₃)₂ | H | H | OH | OH | H | 1.0 ± 0.1 | 3.4 ± 0.4 |
| 9f | O(CH₂)₃N¹(CH₃)₃ | H | H | OH | OH | H | 1.2 ± 0.3 | 4.2 ± 0.2 |
| 9g | O(CH₂)₆N¹(CH₃)₃ | H | H | OH | OH | H | 1.4 ± 0.3 | 4.3 ± 0.6 |
| 9h | O(CH₂)₈N¹(CH₃)₃ | H | H | OH | OH | H | 1.0 ± 0.3 | 3.3 ± 0.6 |
| 9i | O-glucosyl | H | H | OH | OH | H | 2.8 ± 0.9 | 4.9 ± 0.3 |
| 9j | O-rutinosyl | H | H | OH | OH | H | 15.7 ± 4.8 | 4.3 ± 0.3 |
| 10b | OCH₃ | H | OH | OH | OH | H | 1.6 ± 0.7 | 5.9 ± 0.7 |
| 10c | OCH₂CH₂OH | H | OH | OH | OH | H | 21.7 ± 6.1 | 3.4 ± 0.2 |
| 10d | O(CH₂)₃N⁺(CH₃)₃ | H | OH | OH | OH | H | 3.8 ± 0.1 | 5.1 ± 0.3 |
| 11a | OH | H | OCH₂CH₂OH | OH | OH | H | 13.8 ± 4.4 | 4.9 ± 0.4 |
| Quercetin | OH | OH | OH | OH | OH | H | 9 | |
| Rutin | O-rutinosyl | OH | OH | OH | OH | H | 10 | |
| Fisetin | OH | H | OH | OH | OH | H | 16 | |
| Kaempferol | OH | OH | OH | H | OH | H | 6 | |
| Myricetin | OH | OH | OH | OH | OH | OH | 10 | |
| Galangin | OH | OH | OH | H | H | H | 6 | |
| triHEQ | OH | OH | OEtOH | OEtOH | OEtOH | H | 85 | |
| Lutcolin | H | OH | OH | OH | OH | H | 17 | |
| monoHER | O-rutinosyl | OH | OCH₂CH₂OH | OH | OH | H | 12.7 ± 3.7 | 3.0 ± 0.2 |

Table 1 shows that at least the compounds of formula 1 are at least as active as monoHER, which is known to be an excellent antioxidant.

The location of substituents R is indicated in accordance with formula Ia; between brackets the conventional position on the flavonoid molecule is indicated (Van Acker et al., Free Rad. Biol. Med. 20, 331-342, 1996).

Experiment 3: Isolated Mouse Left Atrium Model

In order to determine the cardioprotective properties of the compounds in vitro, the protection against doxorubicin-induced negative inotropy was studied, using the isolated mouse left atrium model.

The example compounds were tested for their protective effect on the negative inotropic action of doxorubicin on the isolated mouse left atrium. The testing model has been described earlier (de Jong et al.Ress Comm Chem Pathol Pharmacol 68, 275-289 (1990). In short, left atria were isolated from male Balb/c mice (18-22 g) and stimulated with square wave pulses of 3 ms duration at a frequency of 4 Hz. Incubation with 35 mM doxorubicin causes a decrease in inotropy of 50% (after 1 h). Atria were incubated with 35 μM doxorubicin and 250 μM flavonoid. Inotropy was measured and expressed as % basal contractile force relative to t=0.

TABLE 2
Inhibition of the negative inotropic action of doxorubicin
in the electrically paced mouse left atrium by the example compounds.

| | | | | Atrium |
|---|---|---|---|---|
| Compound | Substituents | | | protection |
| no. | R1 (3) | R4 (5) | R2 (7) | (250 μM) |
| 9a | OH | H | H | NP¹ |
| 9b | OCH₃ | H | H | NP |
| 9c | OCH₂CH₂OH | H | H | NP |
| 9d | OCH₂COOH | H | H | NP |
| 9e | O(CH₂)₃N(CH₃)2 | H | H | NP |
| 9f | O(CH₂)₃N⁺(CH₃)₃ | H | H | NP |
| 9g | O(CH₂)₆N⁺(CH₃)₃ | H | H | NP |
| 9h | O(CH₂)₈N⁺(CH₃)₃ | H | H | NP |
| 9i | O-glucosyl | H | H | 46% |
| 9j | O-rutinosyl | H | H | NP |
| 10b | OCH₃ | H | OH | NP |
| 10c | OCH₂CH₂OH | H | OH | 13% |
| 10d | O(CH₂)₃N⁺(CH₃)₃ | H | OH | 33% |
| 11a | OH | H | OCH₂CH₂OH | NP |
| monoHER | O-rutinosyl | OH | OCH₂CH₂OH | 52% |

¹No protection
The location of substituents R is indicated in accordance with formula Ia; between brackets the conventional position on the flavonoid molecule is indicated (Van Acker et al., Free Rad. Biol. Med. 20, 331-342, 1996.)

The location of substituents R is indicated in accordance with formula Ia; between brackets the conventional position on the flavonoid molecule is indicated (Van Acker et al., Free Rad. Biol. Med. 20, 331-342, 1996).

As shown by table 2, several compounds of the present invention protect against the negative inotropic effect caused by doxorubicin.

Experiment 4: Protection Against Doxorubicin-Induced Cardiotoxicity In vivo.

One compound (9i) was selected to illustrate the antioxidant action of this series of compounds in vivo. The in vivo cardiotoxicity by weekly i.v. injections of doxorubicin was determined by measuring the ST-interval in the ECG. The ECG was detected by means of telemetry (S.A.B.E. van Acker, et al., Br. J. Pharmacol. 115: 1260-1264 (1995)).

Male BALB/C mice were anesthetized with 0.07 ml per 10 g i.p. of a mixture of Hypnorm® (0.315 mg/ml fentanyl and 10 mg/ml fluanisone), Dormicum® (5 mg/ml midazolam) and sterilised water in the ratio 1:1:2. Surgery was performed as described in detail by Kramer et al. (Pharmacol. Toxicol. Meth., 30: 209-215, (1993)). In short, the transmitter was implanted in the peritoneal cavity of each mouse two weeks before the start of the treatment. The leads of the transmitter were sutured subcutaneously in lead II position (the (−) lead at the right shoulder and the (+) lead towards the lower left chest). Animals were treated for 6 weeks followed by a 2 week period of non-treatment. Doxorubicin was administered once/week (4 mg/kg, i.v.), whereas 9i (68 mg/kg, i.p.) was administered 1 h before doxorubicin and every 24 h over the next 4 days.

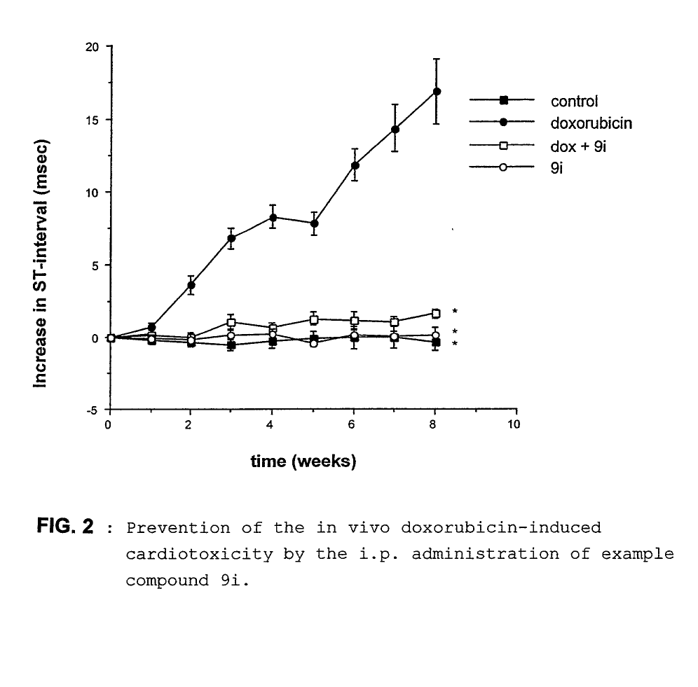

FIG. 2 shows that the example compound has significantly potent activity at the dose of 68 mg/kg, and is at least 5 times more potent as a protector against doxorubicin-induced cardiotoxicity than monoHER, which is known as a cardioprotective agent.

Experiment 5: Interference with the Antitumor Activity of Doxorubicin

Because of the possible use of these compounds as adjuvants in the chemotherapy, it was checked whether the compounds influenced the negative effect of doxorubicin on cell growth. It was found that the newly synthesized compounds did not affect the inhibition on cell growth caused by doxorubicin of three tumor cell lines (OVCAR-3, MCF-7 and A2780). This observation further underlined the use of these compounds as adjuvant therapy in the protection of the side effects of cytostatics.

On day 0 cultured OVCAR-3, MCF-7 and A2780 cells were plated at a density of 5000 cells/well for OCAR-3 and MCF-7 and 3000 cells/well for A2780 cells. After 24 h (day 1) the example compound 9i was added (final concentration 100 μM) in combination with doxorubicin. After incubation of 96 h at 37° C. (day 5), growth inhibitory effects were evaluated using the standard MTT assay.

TABLE 3
Effect on the antitumor activity of doxorubicin

| | Dox | Dox + 9i | 9i 100 μM |
|---|---|---|---|
| Cell line | IC50 (M) | IC50 (M) | growth % |
| A2780 | 3.8 × 10⁻⁹ | 4.5 × 10⁻⁹ | 97.7% |
| MCF-7 | 4.0 × 10⁻⁹ | 4.0 × 10⁻⁹ | 83.8% |
| OVCAR-3 | 6.5 × 10⁻⁹ | 4.8 × 10⁻⁹ | 86.8% |

Table 3 shows that example compound 9i leads to a slight growth inhibition. The combination of 9i with doxorubicin does not reduce the antitumor activity of doxorubicin in vitro.

Experiment 6: Antitumor Activity of Flavonoids

Next to the use of flavonoids as medicaments in free radical mediated diseases, the presence of anti-tumor activity has been reported. Quercetin is the best known example (Molnar et al. Neoplasma, 28: 11-17 (1981)). Because of the possible use of these compounds in chemotherapy, it was checked whether the compounds affected cell growth. It was found that the newly synthesized compounds inhibited the growth of a tumor cell line (OVCAR-3).

On day 0 cultured OVCAR-3 cells were plated at a density of 5000 cells/well. After 24 h (day 1) the example compounds were added and after incubation of 72 h at 37° C. (day 4), growth inhibitory effects were evaluated with the standard SRB (sulforhodamine B) assay (Skehan et al. J. Natl. Cancer Inst., 82: 1107-1112 (1990)).

TABLE 4
Growth inhibition (IC50 μM) OVCAR-3 cells by example
compounds 9a-10b.

| Compound | R2 | R1 | IC50 (μM) |
|---|---|---|---|
| 9a | H | OH | 21.0 ± 2.4 |
| 9b | H | OCH₃ | 2.2 ± 0.4 |
| 9c | H | OCH₂CH₂OH | 44.2 ± 20.1 |
| 10b | OH | OCH₃ | 15.8 ± 1.3 |
| Quercetin | | | 126.0 ± 50.5 |

It was found that the example compounds were more potent than quercetin (a known cytostatic compound) in inhibiting the growth of OVCAR-3 cells.

Experiment 7

Antidiabetic Action

In order to study the antidiabetic action of compound 9i (see FIG. 1 and the section “syntheses” above), the compound was studied in alloxan-induced diabetic rats. Rats that were treated with alloxan (100 mg/kg, iv) showed a threefold increase in plasma glucose level 72 h after alloxan administration (G. Ph. Biewenga et al., Gen. Pharmacol. 29, 315-331 (1997)). These diabetic rats were subsequently treated with compound 9i for two weeks (at 68 mg/kg, i.p.). This led to a decrease in glucose plasma level compared to controls. This experiment therefore demonstrates the antidiabetic action of compound 9i.

Experiment 8

Use in COPD

Chronic Obstructive Pulmonary Disease is associated with oxidative stress. There are strong indications that antioxidant therapy may be beneficial in COPD (J. E. Repine et al., Am. J. Resp. Crit. Care Med. 156, 341-358 (1997)). Not only the number of exacerbations decreases, but also the progressive decrease in lung function becomes less severe under treatment with an antioxidant like N-acetylcysteine. An animal model for COPD is ozone exposure. In contrast to asthma, COPD is characterized by a neutrophil influx. Guinea pigs that are exposed to ozone also show a strong neutrophil influx in the lung as demonstrated by lung lavage (H. J. M. van Hoof, Toxicology 120, 159-169 (1997) and H. J. M. van Hoof et al., Environ. Toxicol. Pharmacol. 5, 69-78 (1998)). We studied the protective effect of compound 9i in this ozone exposure model. It appeared that the pulmonary neutrophil influx could be prevented with compound 9i. To this end compound 9i (at 68 mg/kg, i.p.) was given (24 h, 12 h before ozone exposure and daily during the exposure to ozone/air) to guinea pigs that were exposed to 1 ppm ozone for 12 h which was followed by 12 h of air. The lack of neutrophil influx in the lungs when compound 9i was administered shows the anti-inflammatory action of 9i. It also strongly suggests that compound 9i will be of benefit in the treatment of COPD patients.

What is claimed is:

1. A compound according to formula I, 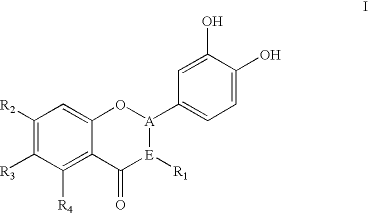
wherein:
A and E form together a C—C or C═C bond,
R₁, R₂, R₃, and R₄ are substituents selected from the group consisting of
H,
OH,
O(CH₂)ₙ-aromatic group, n=0-8,
O(CH₂)ₙ N(CH₃)_{q} with n=0-8, q=0-3,
O(CH₂)ₙ OH with n=1-8,
O(CH₂)ₙ-halide with n=1-8,
O(CH₂)ₙ COOH with n=0-8,
O(CH₂)ₙ COOR′ with n=0-8 and R′ is C₁-C₈ alkyl or an aromatic group,
O(CH₂)ₙ CONH R″ with n=0-8 and R″ is C₁-C₈ alkyl or an aromatic group, and
sugars in mono-, di- or trimeric form or analogues thereof, with the proviso that:
R₁ is not H,
at least two of R₂, R₃ and R₄ are H, and
at most one of R₁, R₂, R₃ and R₄ is OH.

2. A compound according to claim 1, wherein both R₃ and R₄ are H.

3. A compound according to claim 1, wherein at least one of R₁, R₂, R₃ or R₄ are a C₅-C₇ sugar.

4. A compound according to claim 3, wherein the sugar is coupled to E through an —O-linkage.

5. A compound according to claim 3, wherein the sugar is acetylated.

6. A compound according to claim 1, wherein R₁ is an optionally acetylated monosaccharide.

7. A compound according to claim 2, wherein A and E form a C═C bond, and
R₂═H and R₁═OH,
R₂═H and R₁═OCH₃,
R₂═H and R₁═OCH₂CH₂OH,
R₂═H and R₁═OCH₂COOH,
R₂═H and R₁═O(CH₂)₃N(CH₃)₂,
R₂═H and R₁═O(CH₂)₃N⁺(CH₃)₃,
R₂═H and R₁═O(CH₂)₆N⁺(CH₃)₃,
R₂═H and R₁═O(CH₂)₈N⁺(CH₃)₃,
R₂═H and R₁=optionally acetylated O-rutinose
R₂═H and R₁=optionally acetylated O-glucose,
R₂═OH and R₁═OCH₃,
R₂═OH and R₁═OCH₂CH₂OH,
R₂═OH and R₁═O(CH₂)₃N⁺(CH₃)₃, or
R₂═OCH₂CH₂OH and R₁═OH.

8. A compound according to claim 7, wherein R₂═H and R₁ is an O-glucose.

9. A method for preparing of a compound of formula Ia, 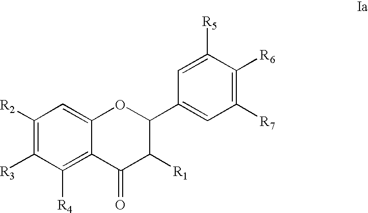
wherein R₁, R₂, R₃, R₄, R₅, R₆ and R₇ are substituents selected from the group consisting of:
H,
OH,
O(CH₂)ₙ-aromatic group, n=0-8,
O(CH₂)ₙ N(CH₃)_{q} with n=0-8, q=0-3,
O(CH₂)ₙ OH with n=1-8,
O(CH₂)ₙ-halide with n=1-8,
O(CH₂)ₙ COOH with n=0-8,
O(CH₂)ₙ COOR′ with n=0-8 and R′ is C₁-C₈ alkyl or an aromatic group,
O(CH₂)ₙ CONH R″ with n=0-8 and R″ is C₁-C₈ alkyl or an aromatic group, and
sugars in mono-, di- or trimeric form or analogues thereof, wherein at least one of R₁-R₇ is selected from the above substituents, and at least one other of R₁-R₇ is OH, comprising reacting a compound of formula II: 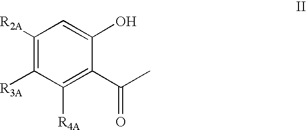
with a compound of formula III: 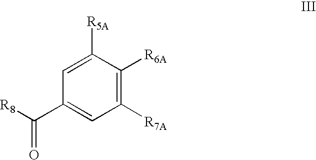
to form a compound with formula IV: 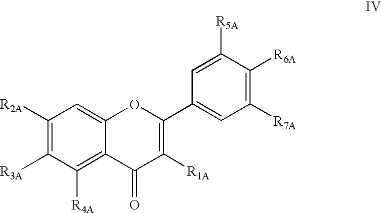
wherein R_{2A}-R_{7A} are, independently from each other, H, OH or the substituent R₂-R₇, respectively, as defined above, R₈ is C₁-C₈ alkoxy, preferably OCH₂CH₃, or H,
R_{1A} is H or OH, and
at least two of R_{1A} of formula IV and R_{2A}-R_{7A} of formulas II and III are OH, wherein prior to said reacting
a) at least one of the OH groups of R_{1A}-R_{7A} of the compounds of formula II, formula III and formula IV are protected with a protecting group, leaving at least one of the OH groups of R_{1A}-R_{7A} unprotected,
b) substituting of at least one of the unprotected OH groups by any of the substituents as defined above, and
c) deprotecting at least one of the OH groups, protected in step a).

10. The method according to claim 9, wherein 2-6 of R₁-R₇ of the compound of formula I are different and at least two of R_{1A}-R_{7A} of the compounds of formula II, III and IV are OH, and at least one of R₁-R₇ is OH, wherein:
a) 2-6 of R_{1A}-R_{7A} are protected by at least two different protecting groups,
b) at least one of the unprotected OH groups are substituted by a first substituent,
c) at least one of the protected OH groups are deprotected, and at least one of the OH groups remain protected, and
further comprising, following step c), for each additional different substituent in R₁-R₇, the following cycle of steps d) and e) are carried out, the number of cycles being equal to the number of additional different substituents of R₁-R₇:
d) substituting OH group or OH groups, deprotected in the preceding step by a substituent different from the substituent(s) of the preceding substitution step,
e) deprotecting at least one of the OH groups, protected in step a).

11. The method according to claim 9, further comprising after the last deprotection step carrying out a final substitution step, substituting the OH group or groups, deprotected at the last deprotection step, by another substituent as defined in claim 9.

12. The method according to claim 9, wherein R₈ in the compound of formula Im is H.

13. A compound according to formula I as defined in claim 1, wherein A-E form a C═C bond,
both R₃ and R₄ are H, and
R₂═H and R₁═OCH₃,
R₂═H and R₁═OCH₂CH₂OH,
R₂═H and R₁═OCH₂COOH,
R₂═H and R₁═O(CH₂)₃N(CH₃)₂,
R₂═H and R₁═O(CH₂)₃N(CH₃)₃,
R₂═H and R₁═O(CH₂)₆N⁺(CH₃)₃,
R₂═H and R₁═O(CH₂)₈N⁺(CH₃)₃,
R₂H and R₁=optionally acetylated O-ratinose
R₂═H and R₁=optionally acetylated O-glucose,
R₂OH and R₁═OCH₃,
R₂═OH and R₁═OCH₂ CH₂OH,
R₂═OH and R₁═O(CH₂)₃N⁺(CH₃)₃, or
R₂═OCH₂CH₂OH and R₁═OH.

14. A compound according to claim 13, wherein R₂═H and R₁ is an O-glucose.

15. The compound according to claim 3, wherein at least one of R₁, R₂, R₃, or R₄ are a C₅ to C₇ mono-, di-, or trisaccharide.

16. The compound of claim 6, wherein R₁ is an optionally acetylated monosaccharide selected from the group consisting of glucose, rhamnose, and fructose.

17. A composition comprising the compound of claim 1 and a pharmaceutically acceptable carrier.

18. A method of treating drug-induced toxicity, doxorubicin-induced cardiotoxicity, free radical mediated diseases, lung diseases, cancer, diabetes mellitus, cardiovascular disease, or arteriosclerosis comprising administering to a patient in need thereof the compound of claim 1 in an amount effective to treat said disease or toxicity.

19. The method of claim 18, wherein the disease or toxicity is drug-induced toxicity.

20. The method of claim 18, wherein the disease or toxicity is doxorubicin-induced cardiotoxicity.

21. The method of claim 18, wherein the disease or toxicity is a free radical mediated disease.

22. The method of claim 18, wherein the disease or toxicity is lung disease.

23. The method of claim 18, wherein the disease or toxicity is cancer.

24. The method of claim 18, wherein the disease or toxicity is diabetes mellitus.

25. The method of claim 18, wherein the disease or toxicity is cardiovascular disease.

26. The method of claim 18, wherein the disease or toxicity is arteriosclerosis.

27. A method of inhibiting the growth of cancer cells comprising administering to said cancer cells with an effective amount of the compound of claim 1.

28. A method of treating chronic obstructive pulmonary disease comprising administering to the lungs an effective amount of the compound of claim 1.
